(19) United States
(12) Patent Application Publication
(10) Pub. No.: US 2026/0346300 A1
(43) Pub. Date: Nov. 5, 2026

(54) BIOMINERALIZATION COATING CAPABLE OF BEING RAPIDLY SYNTHESIZED, PREPARATION METHOD THEREFOR AND USE THEREOF

(71) Applicant: Shanghai Maritime University, Shanghai (CN)

(72) Inventors: Tao LIU, Shanghai (CN); Zhangwei GUO, Shanghai (CN); Na GUO, Shanghai (CN)

(73) Assignee: Shanghai Maritime University, Shanghai (CN)

(21) Appl. No.: 19/112,580

(22) PCT Filed: May 11, 2023
(86) PCT No.: PCT/CN2023/093382

§ 371 Date: Mar. 17, 2025

(30) Foreign Application Priority Data
Feb. 28, 2023 (CN) 202310175729.1

(51) Int. Cl. B05D 7/24; B05D 7/14; C12N 1/20; C12R 1/01; C12R 1/125

(52) U.S. Cl. CPC B05D 7/24; B05D 7/14; C12N 1/20; C12R 2001/01; C12R 2001/125

(57) ABSTRACT

The present disclosure relates to a biomineralization coating capable of being rapidly synthesized, and a preparation method therefor and use thereof. The preparation method includes: S1. performing enlarge culture on Pseudoalteromonas atlantica, Pseudoalteromonas lipolytica, Pseudoalteromonas elyakovii, Bacillus subtilis and Sporosarcina sp., and mixing the same to obtain a bacterial culture solution; S2. immersing a disinfected metal material into the bacterial culture solution to obtain a metal material having a microbial film; and S3. adding sodium carbonate into a precursor solution containing calcium chloride, polylactic acid, hyaluronic acid and fulvic acid, mixing the same to obtain a biomineralization nucleation solution, and then placing the metal material having the microbial film into the biomineralization nucleation solution for standing to obtain a biomineralization coating on the metal material.

### TECHNICAL FIELD

The present disclosure relates to the technical field of material science, in particular to a biomineralization coating capable of being rapidly synthesized, and a preparation method therefor and use thereof.

### RELATED ART

Biomineralization is a common phenomenon in nature, which includes forming insoluble salts from inorganic salts such as calcium and phosphorus through chemical reactions under biological regulation and forming mineralization structures with organic substrates. For example, some biological organisms, such as fish scales, nacre and seaweed, have attracted wide attention because of their excellent mechanical properties and various functions. Among them, natural biomineralization materials that basically and mainly include calcium carbonate have aroused the enthusiasm of researchers to design and create novel biomineralization materials because of their potential use in various fields such as oil-water separation, resistance to biological fouling and drag reduction. Up to now, biomineralization materials prepared by biomimetic means have been manufactured. However, most of these preparation methods are not suitable for industrial scale production. The first common method is the direct use of microbial mineralization and includes forming a calcium carbonate layer through microbial induced mineralization, which takes an extremely long time of up to 14 days throughout the process, such as Marine Bacteria Provide Lasting Anticorrosion Activity for Steel via Biofilm-Induced Mineralization and an oil-water separation steel mesh prepared based on calcium carbonate precipitation induced by Bacillus subtilis and a preparation method therefor disclosed in Patent CN 110743204B. Another common preparation method is the use of a chemical method, which has a complex process, a low preparation speed, a long time that is usually more than ten days and a high cost, and brings a great environmental pollution risk due to the use of a large number of chemical reagents, such as Nacre-inspired underwater superoleophobic films with high transparency and mechanical robustness and a method for preparing a calcium carbonate coating disclosed in Patent CN106519747B. Therefore, the development of a surface that can be rapidly prepared, has certain strength and is synthesized in a fast and simple synthetic way to meet practical application needs of many emerging fields, such as corrosion prevention, has a great use value.

### SUMMARY OF INVENTION

The objective of the present disclosure is to provide a biomineralization coating capable of being rapidly synthesized, and a preparation method therefor and use thereof to overcome the defects of time consumption, a high cost and a complex preparation process of the prior art. The spontaneous and rapid formation of a biofilm from microorganisms is used to replace the most time-consuming and cumbersome step of deposition of an organic film in a biomineralization process of a chemical method, and subsequent chemical synthesis of calcium carbonate greatly accelerates a long process of forming a calcium carbonate deposition in a microbial mineralization process, thereby greatly improving the synthesis speed and the anti-corrosion effect.

The purpose of the present disclosure can be achieved through the following technical solutions:

The first technical solution of the present disclosure is to provide a method for preparing a biomineralization coating capable of being rapidly synthesized, including the following steps:
S1. strain culture: inoculating Pseudoalteromonas atlantica, Pseudoalteromonas lipolytica, Pseudoalteromonas elyakovii, Bacillus subtilis and Sporosarcina sp. into a liquid culture medium, respectively, performing culture under conditions of 25-37° C. and 100-130 r/min for 12-48 h, and mixing the same to obtain a bacterial culture solution;
S2. biofilm growth: immersing a disinfected metal material into the bacterial culture solution obtained in the step S1, and performing culture under conditions of 25-37° C. and 100-130 r/min for 18-24 h to obtain a metal material having a microbial film; and
S3. calcium carbonate deposition: adding sodium carbonate into a precursor solution containing sodium chloride, polylactic acid, hyaluronic acid and fulvic acid, mixing the same by stirring at room temperature, adjusting the pH to alkaline, performing filtration to obtain a biomineralization nucleation solution, and then placing the metal material having the microbial film obtained in the step S2 into the biomineralization nucleation solution for standing to obtain a biomineralization coating on the metal material.

Further, in the step S1, the liquid culture medium consists of the following raw materials by mass percentage: 10-25% of beet molasses, 5-15% of corn steep liquor, 5-10% of glucose, 5-10% of bran, 0.05-1.5% of chitin, 0.5-4% of cellulose, 0.15-0.3% of chitosan, 0.2% of (NH₄)₂SO₄, 0.3% of NaNO 3, 0.2% of K₂HPO₄, and 0.3% of NaCl.

Further, in the step S1, an inoculation amount of each bacterial strain in the liquid culture medium is 0.1% of a total volume of the liquid culture medium; and a volume ratio of the Pseudoalteromonas atlantica, the Pseudoalteromonas lipolytica, the Pseudoalteromonas elyakovii, the Bacillus subtilis and the Sporosarcina sp. is 1:2:1:3:1.

Further, in the step S2, the metal material is not limited to the shape and type, and is preferably an aluminum plate or a low-alloy steel sheet material.

Further, in the step S3, a volume ratio of the sodium carbonate to the precursor solution is 1:1.

Further, in the step S3, a concentration of the sodium carbonate is 10-60 mM.

Further, in the step S3, a concentration of the sodium chloride in the precursor solution is 10-60 mM, an addition mass of the polylactic acid is 0.03-0.05% of a total volume of the precursor solution, an addition mass of the hyaluronic acid is 0.02-0.06% of the total volume of the precursor solution, and an addition mass of the fulvic acid is 0.01-0.03% of the total volume of the precursor solution.

Further, in the step S3, the pH is adjusted to 8-10.

Further, in the step S3, conditions of the standing include a standing reaction in a water bath at 20-30° C. for 5-10 h.

The second technical solution of the present disclosure is to provide a biomineralization coating capable of being rapidly synthesized, which is based on the preparation method as described in the first technical solution.

The third technical solution of the present disclosure is to provide use of the biomineralization coating capable of being rapidly synthesized as described in the second technical solution, wherein the biomineralization coating is used in the field of corrosion prevention.

Compared with the prior art, the present disclosure has the following beneficial effects:
(1) Compared with an ordinary simple microbial or chemical method for preparing a mineralization film, the present disclosure has the characteristics that a biofilm is formed on a surface of the metal material by means of microorganisms and the additional biomineralization nucleation solution modified by common acidic molecules. According to a traditional chemical method, the formation of an organic film on the surface of a material requires several precursors under specific reaction conditions, and the precursors are complicated and time-consuming to prepare. However, the microorganisms can quickly form the biofilm containing abundant mineralization nucleation sites on the surface of the material, because the formation of the biofilm not only depends on the bonding of chemical groups, but also is determined by adhesion characteristics of bacteria themselves, and the formation of the biofilm is a common means of the bacteria to provide a good living environment for themselves. Protein and polysaccharide parts in the biofilm are closely related to mineralization. Acidic groups of proteins play the role of mineralization nucleation, while macromolecular polysaccharide substances play a skeleton role on mineralization. Therefore, the biofilm is faster to prepare than the organic film prepared on the surface of a material by the traditional chemical method.
(2) The reason why a simple chemical method takes a relatively long time to prepare a biomateralization material is that after being bound to calcium ions, organic groups on a biofilm need to overcome a great barrier to bind to carbonate ions in a solution, which takes a relatively long time. However, according to the preparation method of the present disclosure, after the biofilm is formed on the surface of the metal material, the amorphous calcium carbonate in the biomateralization nucleation solution is used to directly bind to an anion group of the biofilm, such that the binding time of the calcium ions and the carbonate ions in an initial nucleation process is shortened, a calcium carbonate structure with a calcite crystal form can be quickly formed within a shorter time, and the method is simple and has a lower use cost and a better effect.
(3) The biomineralization coating prepared by the method of the present disclosure has a uniform, dense and stable structure and is an organic-inorganic composite biomineralization coating with an aragonite structure. A typical “brick mud” structure is formed by the calcium carbonate with the calcite crystal form and the biofilm, and has excellent corrosion resistance.
(4) Materials used in the rapid preparation method provided by the present disclosure are all environmentally friendly materials, such as various bacteria, calcium chloride, sodium carbonate, etc., which are harmless to the human body and the environment. Compared with an ordinary chemical method for preparing a mineralization film, the preparation method of the present disclosure has a higher mineralization film forming speed, simpler operation and a lower cost.

### BRIEF DESCRIPTION OF DRAWINGS

FIG. 1 shows an electrochemical impedance diagram of a low-alloy steel sample coated with a biomineralization coating in Example 1.

FIG. 2 shows a surface SEM image of the low-alloy steel sample coated with the biomineralization coating in Example 1.

FIG. 3 shows an XRD component diagram of the low-alloy steel sample coated with the biomineralization coating in Example 1.

FIG. 4 shows an anti-corrosion effect image of the low-alloy steel sample coated with the biomineralization coating in Example 1.

FIG. 5 shows an electrochemical impedance diagram of an aluminum sample coated with a biomineralization coating in Example 2.

FIG. 6 shows a surface SEM image of the aluminum sample coated with the biomineralization coating in Example 2.

FIG. 7 shows an XRD component diagram of the aluminum sample coated with the biomineralization coating in Example 2.

FIG. 8 shows an anti-corrosion effect image of the aluminum sample coated with the biomineralization coating in Example 2.

### DESCRIPTION OF EMBODIMENTS

The present disclosure is described in detail below in combination with the accompanying drawings and specific examples.

In the following various examples, unless otherwise specified, it is indicated that all raw materials or treatment technologies are conventional commercially available raw material products or conventional treatment technologies in the art.

Bacteria in the following various examples were purchased from Marine Culture Collection of China, and the bacteria used were non-pathogenic and were purchased on Nov. 29, 2021, with an order number of 202111291714230140.

The preservation number of Pseudoalteromonas atlantica was MCCC 1G 00119.

The preservation number of Pseudoalteromonas lipolytica was MCCC 1A 00565.

The preservation number of Pseudoalteromonas elyakovii was MCCC 1C 01192.

The preservation number of Bacillus subtilis was MCCC 1K 05317.

The preservation number of Sporosarcina sp. was MCCC 1A 00659.

### Example 1

### (1) Enlarge Culture of Bacterial Strains

Pseudoalteromonas atlantica, Pseudoalteromonas lipolytica, Pseudoalteromonas elyakovii, Bacillus subtilis and Sporosarcina sp. were inoculated into a sterilized liquid culture medium at an inoculation amount of 0.1% by volume percentage, respectively, subjected to shaking culture at 37° C. at 120 r/min for 24 h, and then mixed at a volume ratio of 1:2:1:3:1 to obtain a bacterial culture solution.

### (2) Biofilm Growth

A low-alloy steel sheet material was cleaned with anhydrous alcohol, sterilized under an ultraviolet lamp for half an hour, then placed into the bacterial culture medium, and subjected to shaking culture in a shaker at 37° C. at 120 r/min for 18 h to obtain a low-alloy steel sheet having a microbial film.

### (3) Calcium Carbonate Deposition

50 ml of sodium carbonate (10 mM) was added into 50 mL of a precursor solution containing calcium chloride (10 mM), polylactic acid (0.02% (weight/volume)), hyaluronic acid (0.04% (weight/volume)) and fulvic acid (0.02% (weight/volume)) and stirred at room temperature for 15 min. Then, the pH of the above mixed solution was adjusted to 10 by gradually adding a 2 mM NaOH aqueous solution, and filtration was performed for further purification to obtain a biomineralization nucleation solution. The low-alloy steel sheet having the microbial film was placed into the purified biomineralization nucleation solution for a standing reaction in a water bath at 25° C. for 6 h to complete rapid preparation to obtain a low-alloy steel sheet coated with a biomineralization coating.

The corrosion resistance of the low-alloy steel sheet coated with the biomineralization coating was tested.

Electrochemical impedance spectroscopy test: A three-electrode system was used with a platinum sheet as a counter electrode, a saturated calomel electrode as a reference electrode and the low-alloy steel sheet coated with the biomineralization coating as a working electrode in a test range of 10⁻²-10⁵ Hz. The low-alloy steel sheet coated with the biommineralization coating was cleaned with deionized water for three times, then dried with a hair dryer, placed under a scanning electron microscope and observed whether a calcium carbonate deposition was found, and then subjected to component identification by XRD. A blank group sample (a low-alloy steel sheet not coated with the biommineralization coating) and an experimental group sample (the low-alloy steel sheet coated with the biommineralization coating) were immersed into a 3.5% sodium chloride solution for 7 days to observe the macroscopic corrosion morphology.

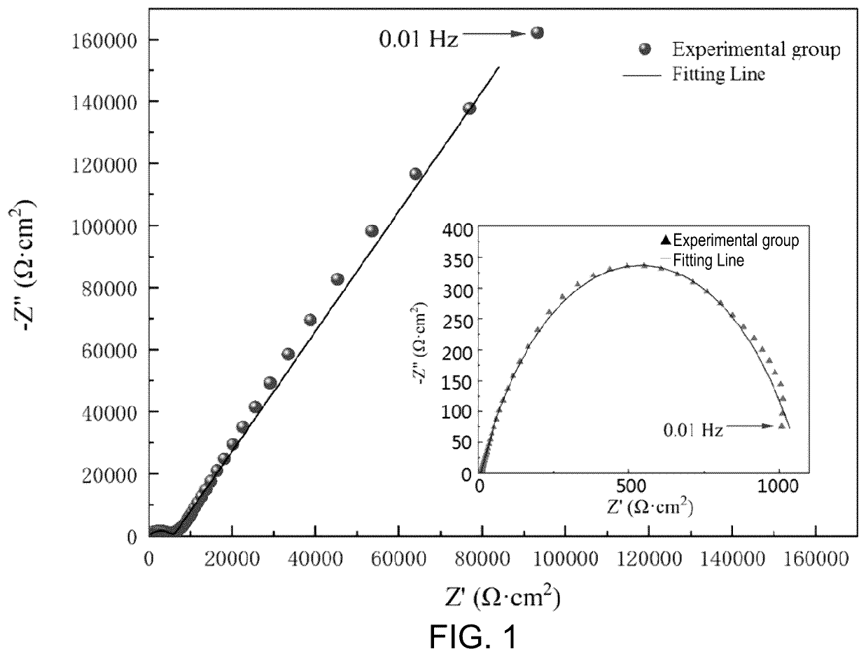

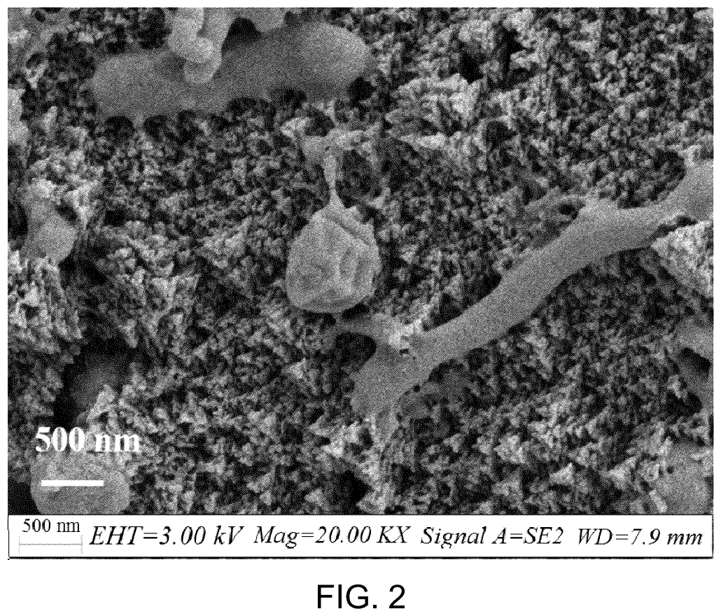

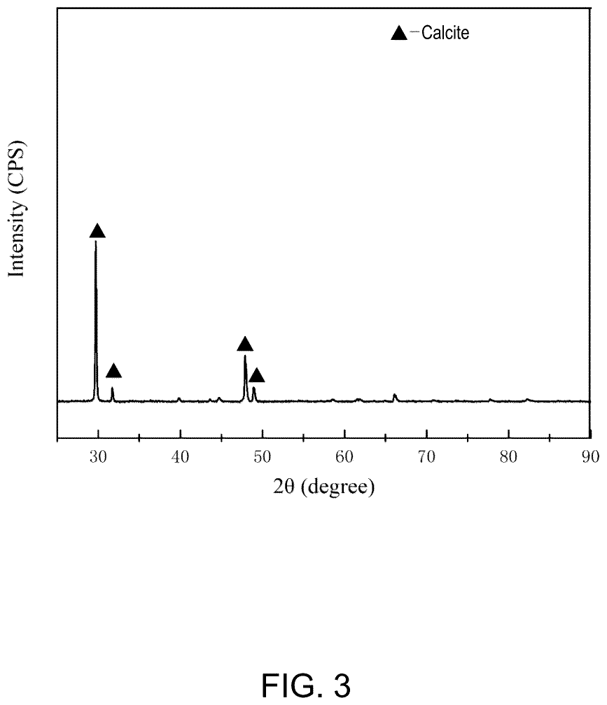

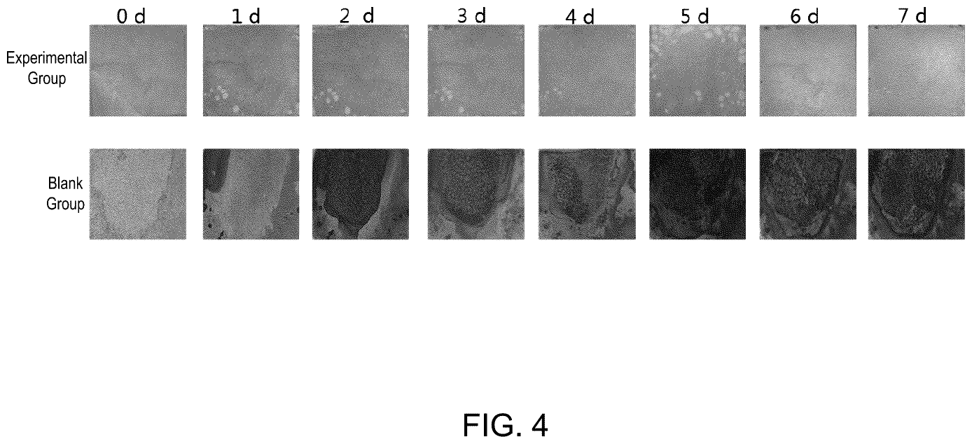

Results were analyzed as follows:
(1) As shown in FIG. 1, electrochemical test results show that an impedance value of the biommineralization coating can reach 10-15 w ohm/cm², while an impedance value of the blank group is much smaller and is only a few hundred ohm/cm², indicating that the biommineralization coating has excellent corrosion resistance.
(2) As shown in FIG. 2, scanning electron microscopy results show that the biomineralization coating is successfully and rapidly prepared on the surface of the low-alloy steel sheet in the present example.
(3) As shown in FIG. 3, XRD results show that the biomineralization coating that is successfully and rapidly prepared on the surface of the low-alloy steel sheet in the present example includes the component of calcium carbonate (i.e., calcite in the figure).
(4) As shown in FIG. 4, macroscopic corrosion morphology results show that the blank group without the cover of the mineralization coating undergoes macroscopic corrosion, while the experimental group with the mineralization coating still maintains the original morphology of the coating. Therefore, the biomineralization coating that is successfully and rapidly prepared on the surface of the low-alloy steel sheet in the present example has excellent seawater corrosion resistance.

### Example 2

### (1) Enlarge Culture of Bacterial Strains

Pseudoalteromonas atlantica, Pseudoalteromonas lipolytica, Pseudoalteromonas elyakovii, Bacillus subtilis and Sporosarcina sp. were inoculated into a sterilized liquid culture medium at an inoculation amount of 0.1% by volume percentage, respectively, subjected to shaking culture at 37° C. at 120 r/min for 24 h, and then mixed at a volume ratio of 1:2:1:3:1 to obtain a bacterial culture solution.

### (2) Biofilm Growth

An aluminum plate was cut into 1 cm×1 cm, cleaned with deionized water and anhydrous ethanol, and then sterilized under an ultraviolet lamp for half an hour. The cut aluminum plate was immersed into the bacterial culture medium, and subjected to shaking culture in a shaker at 37° C. at 120 r/min for 18 h to obtain an aluminum plate having a microbial film.

### (3) Calcium Carbonate Deposition

50 mL of sodium carbonate (10 mM) was added into 50 mL of a precursor solution containing calcium chloride (10 mM), polylactic acid (0.02% (weight/volume)), hyaluronic acid (0.04% (weight/volume)) and fulvic acid (0.02% (weight/volume)) and stirred at room temperature for 15 min. Then, the pH of the above mixed solution was adjusted to 10 by gradually adding a 2 mM NaOH aqueous solution, and filtration was performed for further purification to obtain a biomineralization nucleation solution. The aluminum plate having the microbial film was placed into the purified biomineralization nucleation solution for a standing reaction in a water bath at 25° C. for 6 h to complete rapid preparation to obtain an aluminum plate coated with a biomineralization coating.

The corrosion resistance of the aluminum plate coated with the biomineralization coating was tested.

Electrochemical impedance spectroscopy test: A three-electrode system was used with a platinum sheet as a counter electrode, a saturated calomel electrode as a reference electrode and the aluminum plate coated with the biomineralization coating as a working electrode in a test range of 10⁻²-10⁵ Hz. The aluminum plate coated with the biommineralization coating was cleaned with deionized water for three times, then dried with a hair dryer, placed under a scanning electron microscope and observed whether a calcium carbonate deposition was found, and then subjected to component identification by XRD. A blank group sample (an aluminum plate not coated with the biommineralization coating) and an experimental group sample (the aluminum plate coated with the biommineralization coating) were placed in an acidic salt spray environment for 5 days to observe the macroscopic corrosion morphology.

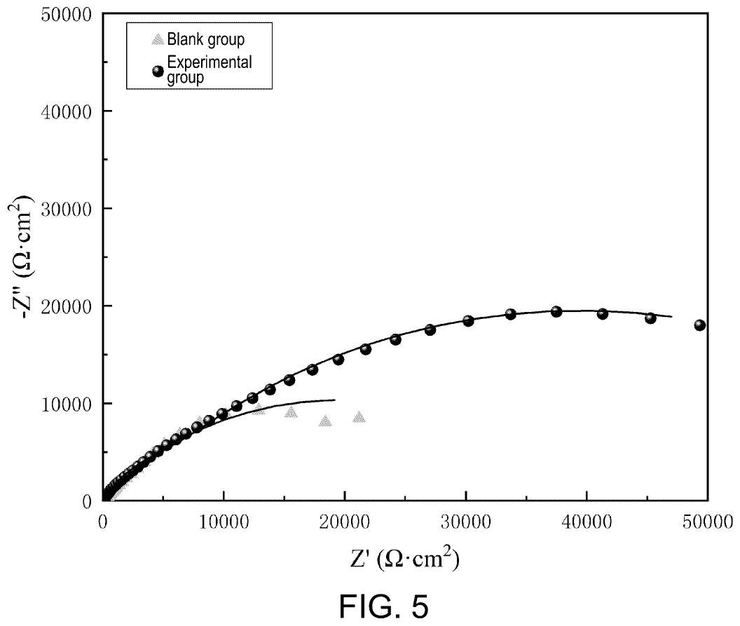

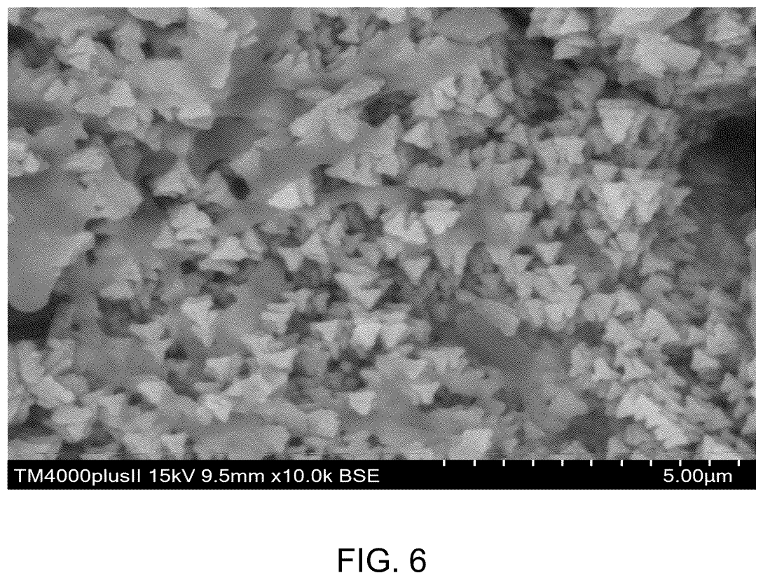

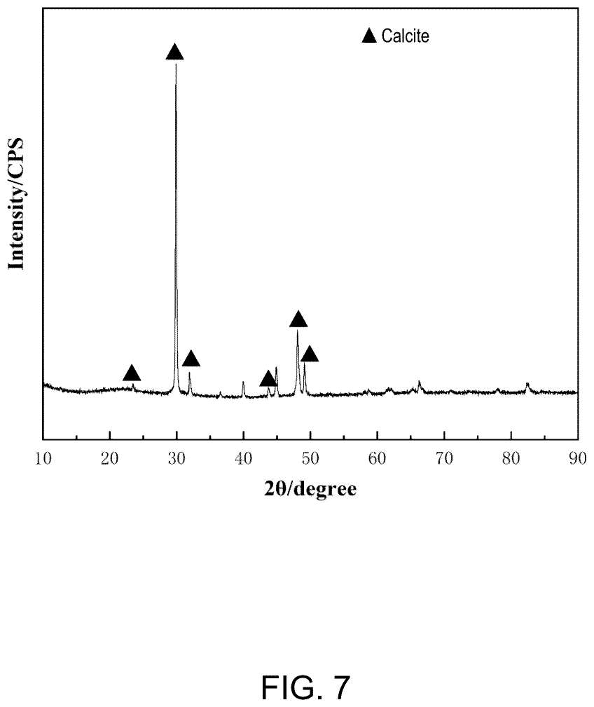

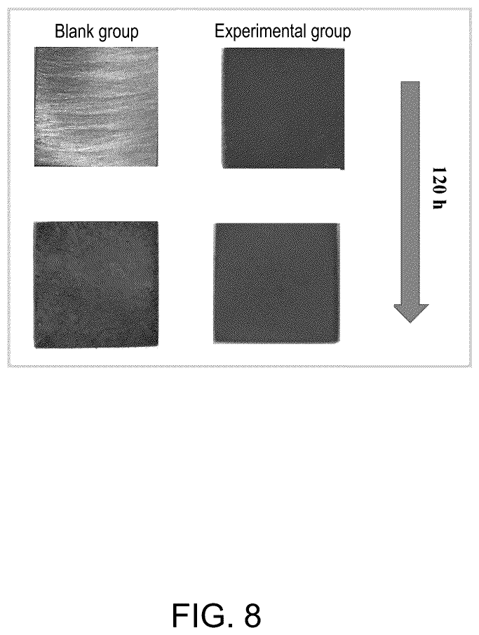

Results were analyzed as follows:
(1) As shown in FIG. 5, electrochemical test results show that an impedance value of the biommineralization coating that is rapidly prepared on the surface of the aluminum plate can reach 3-5 w ohm/cm², while an impedance value of the blank group is much smaller and is only 1-2 w ohm/cm², indicating that the biommineralization coating can improve the corrosion resistance of aluminum to a large extent.
(2) As shown in FIG. 6, scanning electron microscopy results show that the biomineralization coating is successfully and rapidly prepared on the surface of the aluminum plate in the present example.
(3) As shown in FIG. 7, XRD results show that the biomineralization coating that is successfully and rapidly prepared on the surface of the aluminum plate in the present example includes the component of calcium carbonate (i.e., calcite in the figure).
(4) As shown in FIG. 8, macroscopic corrosion morphology results show that the blank group, due to the absence of the mineralization coating, undergoes serious corrosive pitting and is changed from the original bright morphology into dark morphology. Under normal conditions, aluminum remains bright. The phenomena in the figure indicate that the blank group undergoes corrosion, and an oxidation film is destroyed. However, the experimental group, due to the presence of the mineralization coating, has no corrosion after the test while the coating still remains on the surface of aluminum. Therefore, the biomineralization coating that is successfully and rapidly prepared on the surface of the aluminum plate in the present example has excellent acidic salt spray corrosion resistance.

The above description of the examples is intended to facilitate the understanding and application of the present disclosure by those of ordinary skill in the art. It is obvious to those familiar with the art that various modifications of these examples can be easily made, and general principles described herein can be applied to other examples without creative effort. Therefore, the present disclosure is not limited to the above examples, and all improvements and modifications that are made by those skilled in the art based on the disclosure of the present disclosure without departing from the scope of the present disclosure shall fall within the scope of protection of the present disclosure.

1. A method for preparing a biomineralization coating capable of being rapidly synthesized, comprising following steps:
S1. strain culture: inoculating Pseudoalteromonas atlantica, Pseudoalteromonas lipolytica, Pseudoalteromonas elyakovii, Bacillus subtilis and Sporosarcina sp. into a liquid culture medium, respectively, performing culture under conditions of 25-37° C. and 100-130 r/min for 12-48 h, and mixing the same to obtain a bacterial culture solution;
S2. biofilm growth: immersing a disinfected metal material into the bacterial culture solution obtained in the step S1, and performing culture under conditions of 25-37° C. and 100-130 r/min for 18-24 h to obtain a metal material having a microbial film; and
S3. calcium carbonate deposition: adding sodium carbonate into a precursor solution containing calcium chloride, polylactic acid, hyaluronic acid and fulvic acid, mixing the same by stirring at room temperature, adjusting pH to alkaline, performing filtration to obtain a biomineralization nucleation solution, and then placing the metal material having the microbial film obtained in the step S2 into the biomineralization nucleation solution for standing to obtain a biomineralization coating on the metal material.

2. The method for preparing a biomineralization coating capable of being rapidly synthesized according to claim 1, wherein in the step S1, the liquid culture medium consists of following raw materials by mass percentage: 10-25% of beet molasses, 5-15% of corn steep liquor, 5%-10% of glucose, 5%-10% of bran, 0.05%-1.5% of chitin, 0.5%-4% of cellulose, 0.15%-0.3% of chitosan, 0.2% of (NH₄)₂SO₄, 0.3% of NaNO₃, 0.2% of K₂HPO₄, and 0.3% of NaCl.

3. The method for preparing a biomineralization coating capable of being rapidly synthesized according to claim 1, wherein in the step S1, an inoculation amount of each bacterial strain in the liquid culture medium is 0.1% of a total volume of the liquid culture medium; and a volume ratio of the Pseudoalteromonas atlantica, the Pseudoalteromonas lipolytica, the Pseudoalteromonas elyakovii, the Bacillus subtilis and the Sporosarcina sp. is 1:2:1:3:1.

4. The method for preparing a biomineralization coating capable of being rapidly synthesized according to claim 1, wherein in the step S3, a volume ratio of the sodium carbonate to the precursor solution is 1:1.

5. The method for preparing a biomineralization coating capable of being rapidly synthesized according to claim 1, wherein in the step S3, a concentration of the sodium carbonate is 10-60 mM.

6. The method for preparing a biomineralization coating capable of being rapidly synthesized according to claim 1, wherein in the step S3, a concentration of the calcium chloride in the precursor solution is 10-60 mM, an addition mass of the polylactic acid is 0.03-0.05% of a total volume of the precursor solution, an addition mass of the hyaluronic acid is 0.02-0.06% of the total volume of the precursor solution, and an addition mass of the fulvic acid is 0.01-0.03% of the total volume of the precursor solution.

7. The method for preparing a biomineralization coating capable of being rapidly synthesized according to claim 1, wherein in the step S3, the pH is adjusted to 8-10.

8. The method for preparing a biomineralization coating capable of being rapidly synthesized according to claim 1, wherein in the step S3, conditions of the standing comprise a standing reaction in a water bath at 20-30° C. for 5-10 h.

9. A biomineralization coating capable of being rapidly synthesized, wherein the biomineralization coating is based on the preparation method according to claim 1.

10. A use of the biomineralization coating capable of being rapidly synthesized according to claim 9, wherein the biomineralization coating is used in a field of corrosion prevention.

11. A biomineralization coating capable of being rapidly synthesized, wherein the biomineralization coating is based on the preparation method according to claim 2.

12. A biomineralization coating capable of being rapidly synthesized, wherein the biomineralization coating is based on the preparation method according to claim 3.

13. A biomineralization coating capable of being rapidly synthesized, wherein the biomineralization coating is based on the preparation method according to claim 4.

14. A biomineralization coating capable of being rapidly synthesized, wherein the biomineralization coating is based on the preparation method according to claim 5.

15. A biomineralization coating capable of being rapidly synthesized, wherein the biomineralization coating is based on the preparation method according to claim 6.

16. A biomineralization coating capable of being rapidly synthesized, wherein the biomineralization coating is based on the preparation method according to claim 7.

17. A biomineralization coating capable of being rapidly synthesized, wherein the biomineralization coating is based on the preparation method according to claim 8.
